(19) United States
(12) Patent Application Publication
(10) Pub. No.: US 2008/0183077 A1
(43) Pub. Date: Jul. 31, 2008

(54) HIGH INTENSITY FOCUSED ULTRASOUND PATH DETERMINATION

(75) Inventors: Romain Moreau-Gobard, Palo Alto, CA (US); Arif Sanli Ergun, Mountain View, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 11/874,351

(22) Filed: Oct. 18, 2007

Related U.S. Application Data
(60) Provisional application No. 60/852,821, filed on Oct. 19, 2006.

(51) Int. Cl. A61N 7/00

(52) U.S. Cl. 600/439

(57) ABSTRACT

Paths are determined for high intensity focused ultrasound. A subset of possible paths is selected for the application of high intensity focused ultrasound. Obstructions (e.g., bone or metal), tissue characteristics (e.g., organ or tissue sensitivity to heat or attenuation characteristic), distance, or another factor are used to select the scan lines for high intensity focused ultrasound. The selection may be aided by ultrasound imaging data, such as data representing a volume. The response from different regions is used to identify the tissue characteristics or obstructions. The factors may also be used to determine a dose (power) and/or frequency of the high intensity focused ultrasound.

### RELATED APPLICATIONS

The present patent document claims the benefit of the filing date under 35 U.S.C. §119(e) of Provisional U.S. Patent Application Ser. No. 60/852,821, filed Oct. 19, 2006, which is hereby incorporated by reference.

### FEDERALLY SPONSORED RESEARCH OR DEVELOPMENT

The U.S. Government has a paid-up license in this invention and the right in limited circumstances to require the patent owner to license others on reasonable terms as provided by the terms of grant no. DARPA05-01 DBAC awarded by DARPA.

### BACKGROUND

The present invention relates to high intensity focused ultrasound (HIFU). HIFU may be used to treat internal tissues of a patient. HIFU generates heat at a region to be treated. The heat may cause coagulation, tissue stress or breakdown, or other effect.

In one approach, a HIFU transducer probe is designed for insertion within a patient. An incision in made, and the HIFU transducer is moved by hand to the tissue to be treated. HIFU is activated to coagulate blood. More remote HIFU may be used.

The physician may be aided in placement of the HIFU transducer by ultrasound imaging. For example, an imaging transducer is positioned outside of the patient's body, but directed to scan and image the region to be treated. However, the imaging transducer can move independently of the HIFU transducer, limiting the ability to guide placement. The imaging may be limited by bone or other acoustically opaque structure, limiting the imaging region or further restricting placement of the imaging transducer. Since the HIFU and imaging transducers are independent, accurate spatial relationship of the imaged region with the treatment region of the HIFU may be difficult to obtain.

### BRIEF SUMMARY

By way of introduction, the preferred embodiments described below include methods, systems, computer readable media, and instructions for high intensity ultrasound path and transmit characteristic determination. A subset of possible paths is selected for the application of high intensity focused ultrasound. Obstructions (e.g., bone or metal), tissue characteristics (e.g., organ or tissue sensitivity to heat or attenuation characteristic), distance, or other factors are used to select the scan lines for high intensity focused ultrasound. The selection may be aided by ultrasound imaging data, such as data representing a volume. The response from different regions is used to identify the tissue characteristics or obstructions. The factors may also be used to determine a dose (power) and/or frequency of the high intensity focused ultrasound.

In a first aspect, a method is provided for high intensity ultrasound path determination. A plurality of possible paths is identified for high intensity ultrasound from one or more transducers to a treatment region within a patient. A subset of the plurality of possible paths is selected. The high intensity ultrasound is transmitted along the paths of the selected subset.

In a second aspect, a system is provided for high intensity ultrasound path determination. At least one therapy transducer is operable to transmit high intensity focused ultrasound. At least one imaging transducer is operable to transmit acoustic energy for imaging. A processor is operable to determine an origin of a beam of the high intensity focused ultrasound relative to a treatment region. The origin is determined as a function of patient characteristics between origin options and the treatment region. The processor is operable to determine the patient characteristics and origin as a function of data received with the imaging transducers. The imaging and therapy transducers may be a same device.

In a third aspect, a computer readable storage medium has stored therein data representing instructions executable by a programmed processor for high intensity ultrasound path determination. The storage medium includes instructions for transmitting acoustic energy along a plurality of scan lines, receiving signals responsive to the transmitting, and optimizing, for high intensity focused ultrasound, a path to a region to be coagulated, the optimizing being a function of the received signals.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for high intensity ultrasound path determination;

FIG. 2 is a perspective view of a blanket transducer arrangement for ultrasound imaging and high intensity focused ultrasound therapy according to one embodiment;

FIG. 3 is a flow chart diagram of one embodiment of a method for high intensity ultrasound path determination;

FIG. 4 is a graphical representation of one example of path determination;

FIG. 5 is an example medical image showing two selected paths; and

FIGS. 6A-F show example effects for high intensity focused ultrasound beam characteristics.

### DETAILED DESCRIPTION OF THE DRAWINGS AND PRESENTLY PREFERRED EMBODIMENTS

In one example embodiment, multiple transducers are wrapped around or lay along a portion of a patient. By providing the transducers in one device, such as a semi-flexible blanket, movement of the device may be limited or the therapy and imaging transducers may be subject to the same movement, easing alignment of imaging and therapy.

At least some of the transducers in this example are used for volume imaging with ultrasound. As volumes are acquired from each transducer, post-processing software aligns and combines the data representing the scanned volumes to make one complete volume. The volume may have improved image quality because of spatial compounding due to volume overlap. The software and/or user input detect a bleeder or other treatment location. Once the area of interest has been identified in the volume, the software identifies which transducers to use for therapy. Obstructions, tissue characteristics, or other factors are used to select from the possible paths. For example, the volume data is examined to identify desired or undesired acoustic paths. As another example, test transmissions are performed along possible paths, and the returning echo signals indicate the desirability of the tested paths. After selection of one or more paths, a homeostasis beam is transmitted

The acoustic data representing the selected paths may be used to compute the dose to seal the bleeding vessel. The localization information and the power dose are determined from acoustic data. The entire detection of internal bleeding and treatment may be automatic.

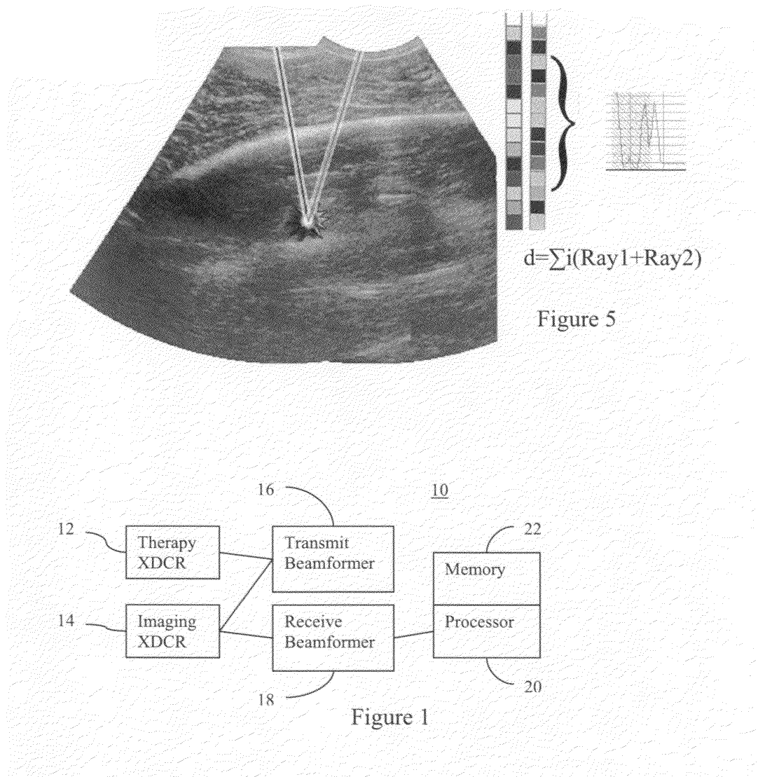

FIG. 1 shows a system 10 for high intensity ultrasound path determination. The system 10 includes a therapy transducer 12, an imaging transducer 14, a transmit beamformer 16, a receive beamformer 18, a processor 20, and a memory 22. Additional, different, or fewer components may be used. For example, the therapy and imaging transducers 12, 14 may be a same device. As another example, more transducers of either type may be provided. In another example, a display is provided. Different transmit beamformers 16 may be used for the different types of transducers 12, 14.

In one embodiment, the system 10 is part of an ultrasound imaging and/or therapy system. The system 10 may be for operation with one or more of the transducers 12, 14 internal or external to the patient. A cart imaging system, computer, workstation, or other system may be used. In another embodiment, the system 10 is portable, such as for carrying by medics, soldiers, emergency response personnel, or others. The portable system 10 weighs from 1-30 kg.

The therapy transducer 12 is any now known or later developed transducer for generating high intensity focused ultrasound from electrical energy. A single element may be provided, such as where focus is provided mechanically by movement or a lens. A plurality of elements in a one or multi-dimensional array may be used, such as an array of N×M elements where both N and M are greater than 1 for electric based focusing or steering.

The element or elements are piezoelectric, microelectromechanical, or other transducer for converting electrical energy to acoustic energy. For example, the therapy transducer 12 is a capacitive membrane ultrasound transducer.

The therapy transducer 12 is operable from outside a patient. For example, the therapy transducer 12 is a probe or other device held against the patient's skin. The therapy transducer 12 is handheld, positioned by a device, or strapped to the patient. In other embodiments, the therapy transducer 12 is in a probe, catheter or other device for operation from within a patient.

In one embodiment, only one therapy transducer 12 is provided. In other embodiments, a plurality of therapy transducers 12 is provided. For example, a plurality of two-dimensional arrays of elements is used for transmitting from different locations to a treatment region.

The imaging transducer 14 is the same or different type, material, size, shape, and structure than the therapy transducer 12. For example, one or more imaging transducers 14 each include a multi-dimensional array of capacitive membrane ultrasound transducer elements. The imaging transducer 14 is any now known or later developed transducer for diagnostic ultrasound imaging. The imaging transducer 14 is operable to transmit and receive acoustic energy.

Where the imaging and therapy transducers 12, 14 are different devices, the spatial relationship between the transducers 12, 14 is measurable. For example, pairs of the imaging and therapy transducers 12, 14 are fixedly connected together or a sensor measures the relative motion between the two. Any sensor may be used, such as magnetic position sensors, strain gauges, fiber optics, or other sensor. Alternatively or additionally, acoustic response from the arrays indicates the relative positions. Correlation of imaging data may indicate spatial relationship between imaging transducers 14. In other embodiments, the same array is used for both therapy and imaging.

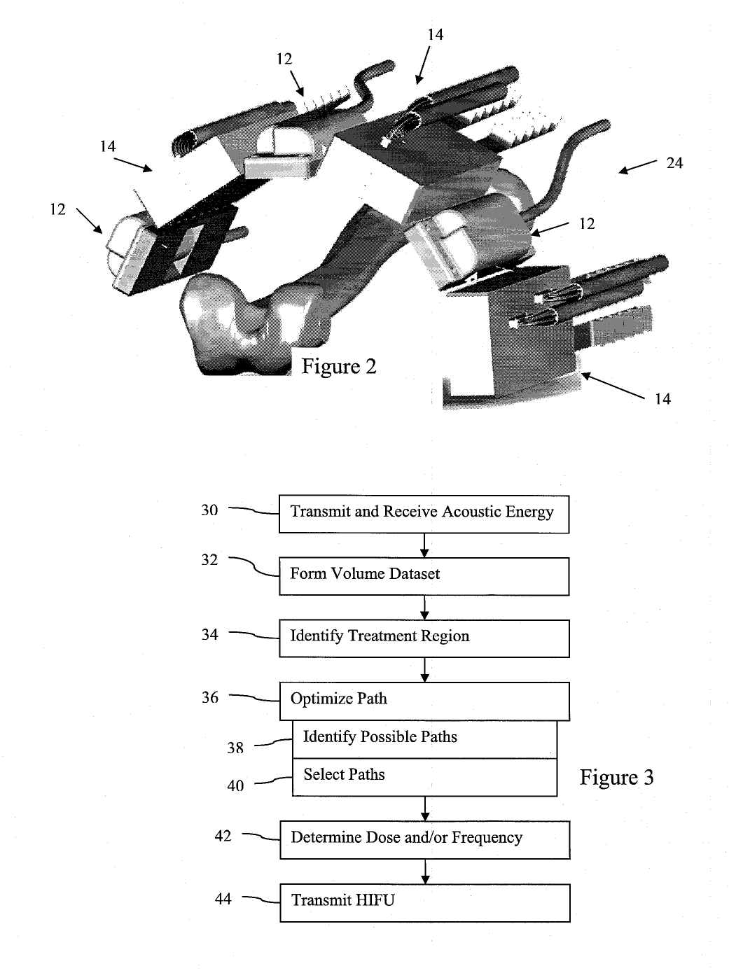

In one embodiment, the therapy and imaging transducers 12, 14 are in a blanket 24. The blanket 24 is plastic, metal, fabric, or other material for rigidly, semi-rigidly or flexibly holding the plurality of transducers 12, 14 with or without the beamformers 16, 18, and/or processor 20. For example, FIG. 2 shows a blanket 24 with a plurality of transducers 12, 14. Hinges, other structure, or an outer casing interconnect the transducers 12, 14. For example, hinges connect the transducers 12, 14. One or more sets of transducers may be more rigidly connected.

The blanket 24 includes every other transducer as an imaging transducer 14 and a therapy transducer 14. Other ratios and/or arrangements may be provided. One, more, or all of the transducers may be dual use devices, such as each transducer 12, 14 being for imaging and therapy. In one embodiment, each of the imaging transducers 14 is operable to electronically or electronically and mechanically scan in three dimensions for acquiring data representing a volume. The transducers 14 may be arranged such that, at least for deeper depths, the scan volumes of adjacent imaging transducers 14 overlap.

A covering, such as a fabric, plastic or other material, may relatively connect the transducers 12, 14. The blanket 24 is a cuff or other structure for wrapping around or resting on a patient. FIG. 2 shows the blanket 24 of transducers 12, 14 wrapped at least partially around a leg or arm. The ultrasound devices are embedded in a flexible surface, wrapped around the region of the body needing medical attention. This geometry may allow acquiring 360-degree images around a limb or larger volumes than with a single array.

The transmit beamformer 16 has a plurality of waveform generators, amplifiers, delays, phase rotators, and/or other components. For example, the transmit beamformer 16 has waveform generators for generating square or sinusoidal waves in each of a plurality of channels. The waveform generators or downstream amplifiers set the amplitude of the electrical waveforms. For imaging, the amplitude is set to provide scanning with acoustic beams below any limits on imaging amplitude. The amplitude may be set for high intensity focused ultrasound, such as higher than associated with imaging.

Relative delays and/or phasing of the waveforms focus the transmitted acoustic energy. By applying relatively delayed and/or apodized waveforms to different elements of a transducer, a beam of acoustic energy may be formed with one or more foci along a scan line. Multiple beams may be formed at a same time. For electronic steering, the relative delays establish the scan line position and angle relative to the transducer 12, 14. The origin of the scan line on the transducer 12, 14 is fixed or may be adjusted by electronic steering. For example, the origin may be positioned on different locations on a multi-dimensional array. The different origins result in different positions of the respective scan lines.

The receive beamformer 18 receives electrical signals from the imaging transducer 14. The electrical signals are from different elements. Using delay and sum beamforming, fast Fourier transform processing, or another process, data representing different spatial locations in a volume is formed. One, a few, or many transmission and reception events may be used to scan a volume with the imaging transducer 14. For example, plan wave transmission and reception is used for scanning a volume. Multiple beam reception with or without synthetic beam interpolation speeds volume scanning with delay and sum beamformation. In alternative embodiments, a two-dimensional plane or scan lines are scanned instead of a three-dimensional volume.

The beamformed data is detected. For example, B-mode detection is provided. In another example, Doppler power, velocity, and/or variance are detected. Any now known or later developed detection may be used. The detected data may be scan converted, remain formatted in the scan format (e.g., polar coordinate), interpolated to a three-dimensional grid, combinations thereof, or in another format. The detection and/or format conversion are done by separate devices, but may be implemented by the processor 20.

The processor 20 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for determining a path for high intensity focused ultrasound. The processor 26 is a single device or multiple devices operating in serial, parallel, or separately. The processor 26 may be a main processor of a computer, such as a laptop or desktop computer, or may be a processor for handling some tasks in a larger system, such as in an imaging system.

The processor 20 determines one or more paths to be used for high intensity focused ultrasound. For example, scan lines appropriate or more desired for the therapy transmissions are determined. The origin of the therapy beam of the high intensity focused ultrasound is identified. The origin and the treatment region define a scan line for transmitting the beam to the treatment region. By moving the origin, different scan lines or paths are identified. The origin may be for different transducers and/or different locations on a same transducer.

The origin and path are determined as a function of any desired factor. In one embodiment, patient characteristics between origin options and the treatment region are used to select the desired origin or origins. The characteristics of the patent along the possible paths are determined from data received with the imaging transducer 14. The imaging data indicates tissue characteristics. The processor 20 uses image processing to determine the tissue characteristics. The data along the paths may be analyzed for variation (e.g., high intensity followed by very low intensity indicating bone or metal with acoustic shadow), lack of variation (e.g., no tissue boundary), threshold intensity (e.g., bone or metal), Doppler response (e.g., fluid region), or other information. Two or three-dimensional processes, such as filtering and classification, may be used to identify tissue regions, tissue type, or other tissue characteristic.

Any patient characteristic may be used, such as tissue attenuation, tissue type or identity, bone structure, metal fragments (e.g., shrapnel, bullet, or medical equipment), fluid region, or tissue boundaries. For example, bone or metal are identified as the patient characteristic along or adjacent to one or more possible paths. Instead of attempting to transmit high intensity focused ultrasound through or by acoustically opaque or scattering objects, other paths without such obstructions are selected. For example, therapy transducers or origins on a same therapy transducer without an obstruction along the path are selected. The selected origin or origins avoid transmitting the high intensity focused ultrasound through the bone or metal. The selected origin is from one of a plurality of therapy transducers or origins on a same transducer and not from another of the plurality of therapy transducers or origins on the same transducer.

Paths with more fluid may be selected, since fluid may be better able to disperse any heat generated by even the distributed or out of focus high intensity ultrasound. A path through or by heat sensitive tissue may not be selected. Paths associated with less attenuation due to distance and/or tissue type may be selected. Paths with less scattering, such as with fewer tissue boundaries, may be selected.

The processor 20 may determine a power, frequency, or other characteristic of the transmitted high intensity focused ultrasound. The patient characteristic between the origin and the treatment region is used to set the power. Greater attenuation due to distance or tissue type may be accounted for by increasing the power. Greater scattering may be accounted for by increasing the power. The frequency may adapt depending on the type of transducer, depth, attenuation along the path, or other characteristic.

The memory 22 stores the ultrasound data for image processing. Alternatively or additionally, the memory 22 stores instructions for programming the processor 20 for high intensity ultrasound path determination and transmit characteristics. The instructions for implementing the processes, methods and/or techniques discussed above are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

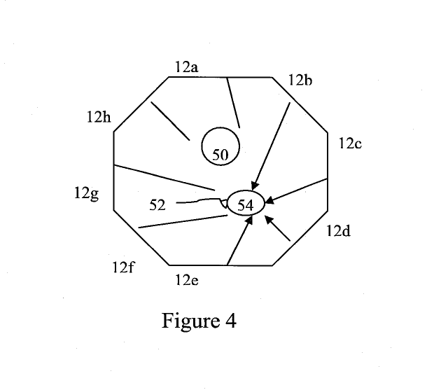

FIG. 3 shows a method for high intensity ultrasound path and transmit characteristic determination. The method uses the system 10 of FIG. 1, the blanket 24 of FIG. 2, different transducers, and/or different systems. The acts are performed in the order shown or a different order. Additional, different, or fewer acts may be used. For example, the method is performed without act 32 by transmitting along possible paths rather than forming a volume data set.

In act 30, acoustic energy is transmitted along a plurality of scan lines, and echoes are received in response to the transmission. The received echoes are converted into received electrical signals. The transmission and reception are performed for imaging and/or testing possible paths.

The scan lines correspond to possible paths. For example, the transmit and receive beams are formed along scan lines intersecting the region to be coagulated and from available sources of the high intensity focused ultrasound. One or multiple arrays may be used to form the beams along the desired scan lines. Previous imaging or other sensing may be used to determine the location of the region to be treated relative to the transducer or transducers.

Alternatively, the scan lines are formatted for scanning a plane or volume regardless of the possible paths. In one embodiment, a dataset representing a three-dimensional volume is formed by transmitting and receiving. The dataset is formed by scanning an entire volume. Alternatively, different scans of overlapping volumes are performed, and the overlapping volumes are combined. Different transducers scan different, but overlapping volumes.

In act 32, a volume dataset is formed. The volume dataset may be formed by scanning a volume with an array, or by combining datasets representing different volumes. Alternatively, a planar dataset is formed of data representing one or more planes, but not an entire volume.

In one embodiment, a stitching or “mosaicking” operation combines different volumetric datasets. For example, a first volume is expanded or added to with each new volumetric acquisition, while assuring insertion of the new information at the correct spatial position. In one embodiment, an ultrasound blanket device performs an initial acquisition, taken as reference. Then, additional volumes are acquired for combination.

The overlapping volumes are aligned. Position sensors, data correlation, or combinations thereof are used to determine the relative spatial position of the overlapping volumes. For correlation, speckle or features may be used. In one embodiment, power Doppler information is segmented to identify one or more surfaces in each data set. The surfaces are then correlated by searching different rotations and/or translations. The relative position with the highest or sufficient correlation indicates the proper alignment. Cross-correlation, minimum sum of absolute differences, or other correlation may be used.

In other embodiments, B-mode data is used for alignment. In another embodiment, the power Doppler based alignment is refined by further B-mode alignment. The power Doppler provides a lower resolution alignment, and the B-mode provides a higher resolution alignment. Features, speckle, segmentation, or other processes are used for B-mode alignment. For example, B-mode data with or without spatial filtering is correlated without specific feature extraction. In yet another embodiment, position sensor information or known spatial limitations of the relative position of the transducers (e.g., semi-rigid connection between transducers) is used to limit the search space for correlation. Any search technique may be used, such as set searching, numerical optimization, coarse-fine, or other.

The data of the aligned volumes is combined. The information is merged with the previous scan, based on the known mutual location of the transducers or volumes. Any combination may be used, such as selecting a datum for each spatial location from available datasets, averaging, weighted averaging to avoid combination artifacts, or interpolation. The aligned and combined volumes provide a larger three-dimensional volume.

The volume dataset may be used for three-dimensional position determination and/or to generate cut planes, such as multiplanar reconstruction. For example, a cut plane, which intersects and is co-axial with a vessel, is formed for identifying a region to be treated.

In act 34, the region to be coagulated or treated is identified. Manual, automatic, or semi-automatic identification is used. For example, the user selects a point in different views as indicating the location of a bleeding vessel. The geometric relationship of the different views may provide an indication of a location in a volume for treatment. As another example, a processor identifies the region for vascular closure of an internal hemorrhage. An image process is performed to identify the leakage of a vessel. The volume dataset or other data representing the possible location of internal bleeding is processed. Any type of data may be processed, such as ultrasound, CT, X-ray, or MRI.

In one embodiment, ultrasound data representing the volume, such as acquired with a blanket ultrasound device, is used to localize the bleeder with a processor. For example, Doppler information shows a flow pattern associated with bleeding. As another example, B-mode data shows a tear or hole in a vessel wall using boundary detection and high pass filtering of the boundary. In another example, power Doppler data is segmented to identify the locations of flow within a volume. Using skeletonization, the centerlines of any flow and bifurcations result. The bifurcation represents either a vessel branch or bleeding. Spectral Doppler gates are position in the vessel before a bifurcation and at each branch of the bifurcation. Alternatively, a spectral Doppler gate is positioned at only one branch. The systole and diastole spectral response patterns for vessel flow and bleeding are different. Bleeding has less heart cycle variation. By examining a pattern or by comparing patterns, the processor may determine whether a bifurcation is a hemorrhage. In yet another example, acoustic force radiation is used to vibrate a vessel wall. Differences in vibration results may indicate a location of bleeding.

In act 36, a path to the region to be coagulated is optimized for high intensity focused ultrasound. The path is optimized by selecting a better path than others.

A plurality of possible scan lines is determined in act 38. For example, scan line origins based on the available transducers for HIFU and/or based on the available or sampled locations on the face of one or more transducers for HIFU are included in the set of possible paths. For example, two or more scan lines are identified as originating from a respective two or more separate therapy transducers. Other limitations or inclusions may be used to determine the set of possible paths. Each path is a straight line from the origins to the region to be treated within the patient, so corresponds to a scan line or beam volume for the transmission of an ultrasound treatment beam.

The optimization provides for one or more paths. For example, multiple paths may be used to distribute a heat load on skin or tissue not to be treated. A single path may be used.

The spatial relationship of the HIFU transducers to the location to be treated is known or measured. For example, each HIFU transducer is rigidly mounted to an imaging transducer. The alignment of data from the different imaging transducers and the use of imaging data to identify the treatment region provide the spatial relationship of the HIFU transducer to the treatment region. As another example, the relative position of the HIFU transducer to the imaging transducer is measurable, such as with a strain gauge or other sensor. In another example, acoustic reflections from the HIFU transducer indicate the spatial relationship of the HIFU transducer to an imaging transducer. Combinations of these techniques or other techniques may be used.

In act 40, one or more of the possible paths are selected. All or a subset of one or more of the possible paths are selected. The optimization is a function of the received signals. Signals received from scanning the treatment region and around the treatment region indicate the path or paths to be used.

In one embodiment, the possible paths are tested by transmitting along each possible path. Acoustic energy, such as for imaging, is transmitted along the scan lines of each possible path. The signals representing the returning echoes along the scan lines are examined to identify the optimum path or paths.

In another embodiment, the possible paths are identified through a volume where the data representing the volume is acquired without regard to the possible paths or aligned with the possible paths. The scan lines for acquiring the volume dataset may or may not correspond to the possible paths. Since data is acquired for the volume, at least a portion of each possible path has data representing the path. Rays corresponding to the possible paths are cast through or positioned within the volume. The rays are from the available sources of the HIFU through the volume to the region to be treated. The data along the rays may be examined for optimization.

Once the overlapping volumes are stitched together, it is possible to retrieve any voxel (data from the dataset). For a given voxel along the ray, the imaging transducers which contributed data for the combination (e.g., for selection or averaging) are identified. A list of ultrasound imaging transducers involved for any particular point in the medical volume is retrieved. The list of imaging transducer may be used to rule out HIFU transducers associated with imaging transducers that did not contribute data to each voxel along a possible path. For example, an obstruction may result in an imaging transducer not providing data for a location. Alternatively, the data without consideration to source is examined for selection.

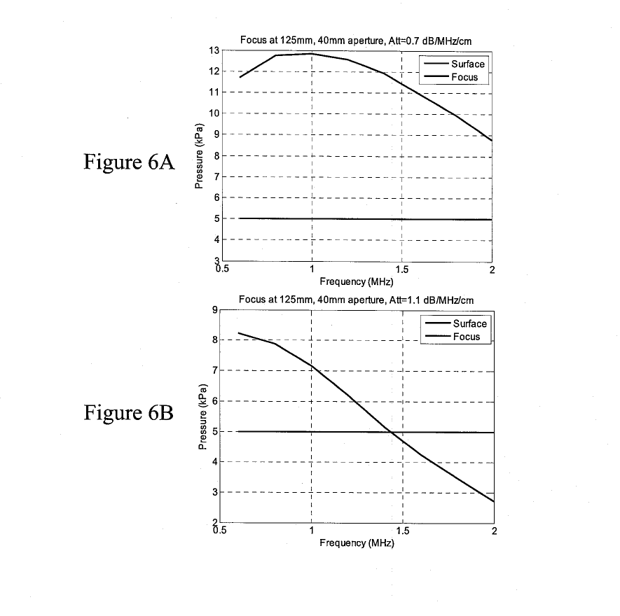

The paths are selected to avoid an acoustic obstruction, a heat sensitive region, a high attenuation region, scatters, or combinations thereof. The characteristics for the selection are provided by the data along the paths. FIG. 4 shows HIFU transducers 12a-h surrounding a treatment region 54. Adjacent the treatment region is a bone 50 and a piece of metal 52, such as associated with hemorrhaging due to shrapnel in a leg. Possible paths are represented by lines from each HIFU transducer 12a-h towards the treatment region 54. For HIFU transducers 12a, and 12f-h, the lines intersect or are close to the metal 52 or bone 50. To provide the desired power for coagulation, the HIFU should not be transmitted into an obstruction. To prevent heating material that may cause further damage (e.g., the metal 52), paths intersecting or close to the material are not selected. The paths free of obstruction are selected, such as from HIFU transducers 12b-e.

Other or different criteria may be used. For example, tissue along a path is heat sensitive, so the path is not selected. As another example, a path passes through more fluid and/or tissues with less attenuation, so is selected. In another example, paths with shorter distances are selected to minimize attenuation, allowing transmission of less power to provide the same power absorption at the treatment region.

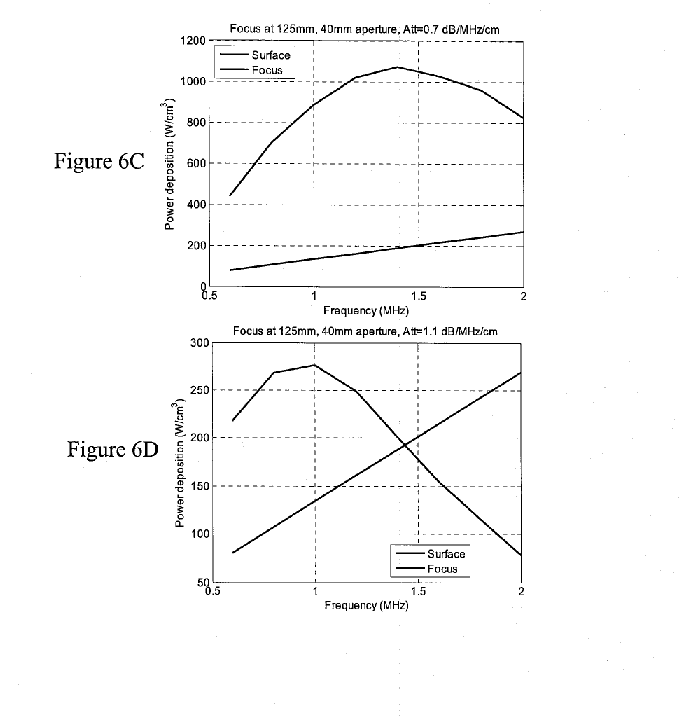

The tissue characteristics (e.g., obstructions) may be detected from the data. Image processing may identify a type of tissue, providing indication of attenuation coefficient. Intensity of reflection, change in intensity as a function of depth or other data analysis indicates obstructions. For example, ray casting in a volume identifies imaging transducers contributing to a voxel at the treatment region. If an imaging transducer did not contribute to the voxels at the treatment region, an obstruction may be indicated. As another example, FIG. 5 shows two rays through a volume or along a plane. The intensities of the voxels along the two rays are shown by voxel and as an analog wave. The intensity variation, peaks, minimum, or other characteristics may indicate the path as desirable or not.

In act 42, the characteristics of the HIFU transmit beam or beams are determined by a processor, by a user, or combinations thereof. The characteristics include power, frequency, combinations thereof, and/or other characteristics (e.g., duration, sequence, or pulse repetition interval). The determination may be a function of the selected paths. For example, higher power pulses may be transmitted for a fewer number of paths. The determination is a function of the desired therapy or amount of power to be delivered in a specific period to cause coagulation or provide treatment. Any now known or later developed dosage considerations may be used for the HIFU beam or beams.

In one embodiment, the power and frequency of the high intensity focused ultrasound is determined, at least in part, as a function of a characteristic of the path. For example, the frequency of the high intensity ultrasound adapts as a function of depth from the HIFU transducer to the treatment region, attenuation characteristic along the path, or combinations thereof. The optimum HIFU frequency depends on the target depth, attenuation constant, the transmit transfer function of the transducer, and any limiting factor. Limiting factors may include, for example, maximizing the power absorption at the target depth or minimizing the power absorption at the skin. The frequency at which the acoustic intensity is highest may not be the optimum HIFU frequency because of the frequency dependence of the acoustic absorption. A desired or optimum HIFU frequency may be calculated given the target depth, and the tissue type between the target and the transducer. Image processing, thresholding, or other technique may be used to distinguish tissue type. For example, fluid, soft tissue and bone tissue types or structures may be distinguished. More subtle distinctions between types of soft tissue may be made. The different types are associated with different acoustic attenuation.

Tissue heating is achieved by absorption of acoustic power. Acoustic absorption is proportional to an attenuation coefficient. Higher attenuation provides higher acoustic power absorption and heat generation. Attenuation and absorption increase with frequency, so it is desirable to use higher frequencies for heating. However, higher propagation attenuation at higher frequencies means shallower penetration depth. There is a trade-off between penetration depth and frequency, and heat. For a given depth of the treatment region, there may be a better frequency at which maximum power deposition (so ΔT) is achieved.

For a plane wave, the pressure at a depth z is related to the pressure at the surface of the transducer with the following equation:

P(z)=P₀·e^{-α·f}^{^{k}}^{·z},

P (z) is the pressure amplitude as a function of depth (z), P₀ is the pressure at z=0, and α·f^{k} is the frequency dependent tissue attenuation constant (k usually takes a value between 1 and 2 depending on the tissue). The acoustic power absorbed by the tissue, L (z), is then calculated as:

$$L ( z ) = \frac{α \cdot {f}^{k}}{{Z}_{0}} {P}^{2} ( z )$$

Absorbed power is proportional to the frequency dependent attenuation constant. The frequency where maximum acoustic power absorption is achieved:

$${f}_{max} = {( \frac{1}{2 \cdot α \cdot z} )}^{\frac{1}{k}}$$

The optimum frequency depends on the depth and attenuation constant. Note that, this calculation is for simple plane waves and is intended to show the dependence of the optimum frequency on the depth and attenuation constant. HIFU beams may be transmitted as a plane wave or with a greater focus. For a transducer with transmit beamforming and a non-uniform tissue type between the transducer and the target (e.g., non-uniform attenuation constant), the optimum frequency may be calculated numerically.

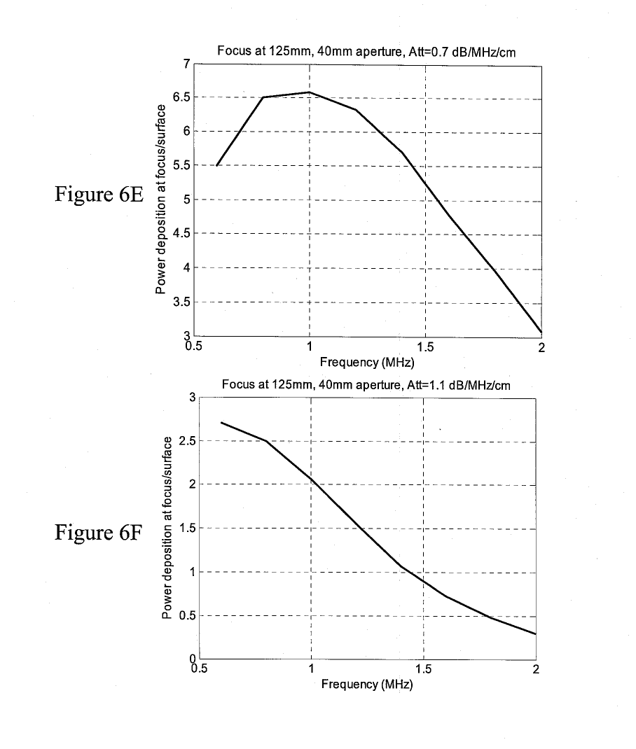

For example, a hypothetical 2D transducer array can generate 5 kPa at its surface independent of the frequency. The transducer has an aperture of 40 mm by 40 mm. FIGS. 6A and 6B show the pressure at the target depth of 125 mm together with the pressure at the surface for 0.7 dB/MHz/cm attenuation (k=1) and for 1.1 dB/MHz/cm attenuation (k=1), respectively. The skin surface is represented by a straight line and the focus is represented by the curve in the Figures of FIG. 6. FIGS. 6C and 6D show the power absorbed by the tissue at the target depth of 125 mm and at the surface for 0.7 dB/MHz/cm attenuation (k=1) and for 1.1 dB/MHz/cm attenuation (k=1), respectively. Comparing FIGS. 6A and 6B with 6C and 6D shows that the frequency of maximum power absorption is not the same as the frequency of maximum acoustic intensity (square of acoustic pressure).

The absorption depends on the attenuation constant. Knowing an average tissue attenuation or the tissue attenuation profile between the target and the transducer may increase the accuracy of optimum frequency calculation. The attenuation constant of different detectable tissue types may be determined and incorporated into the algorithm.

FIGS. 6E and 6F reveal that the operating frequency should be chosen to avoid heating the skin more than the target tissue. Depending on the limiting factor (power absorption at the target depth or power absorption at the skin), the optimum HIFU frequency may be different.

This example shows the case for a transducer with a flat spectral bandwidth. The transducers may have a transfer function affecting the optimum frequency. For a given transducer whose transmit transfer function is known and a given target depth from the transducer, if the attenuation constant is known in the tissue between the transducer and the target, then the optimum HIFU frequency can be calculated numerically. The HIFU optimum frequency can be calculated automatically. The frequency may depend on fewer or additional factors, such as just on the attenuation.

In addition or as an alternative, the power dose of the high intensity ultrasound along each of the selected paths is determined. The power dose may be determined a function of tissues along the path, distance from the transducer to the treatment region along the path, number of paths in the subset, frequency of the transmission, combinations thereof, or other factors. For example, different tissue types provide different attenuation. The different attenuation of the treatment region and the regions between the treatment region and the transducer may alter the power delivered for treatment. Greater attenuation along the path may result in a higher power dose transmitted from the transducer. Greater absorption at the treatment region may result in a less power dose transmitted from the transducer. The power dose is altered by changing frequency, amplitude, or number of cycles of the transmitted waveforms.

The specific tissue types may be identified. Alternatively, the intensity of the echoes or data along the path may indicate tissue characteristics. For example, FIG. 5 shows the intensities along two paths. By collecting the intensities along the paths, the amount of power to reach that particular anatomical point with a desired power level is calculated. The average intensity, sum of intensities, or intensity profile may correlate with attenuation. Other functions may be used to determine power dose.

In act 44 of FIG. 3, the high intensity ultrasound is transmitted along one or more of the paths of the selected subset of possible paths. The high intensity focused ultrasound is transmitted along the selected rays or scan lines. The HIFU transmit beam has a greater cumulative power than the imaging acoustic energy. For a given beam, the power of the HIFU may be greater than used for the imaging beams. If sufficient paths are provided, the HIFU power for a given beam may be less due to distribution of the transmitted power. Since the HIFU beams have the same or adjacent focus, the delivered power at the treatment region is greater than from an imaging scan.

The HIFU beams are transmitted along each path at a same or substantially same time so that the power delivered at a given time at the treatment region is sufficient. Sequential transmission along different or the same paths or combinations of sequential and simultaneous may be used to provide the desired total power, temporally distributed power, and/or spatially distributed power.

The ultrasound energy is focused at the treatment region. If sufficient energy is radiated to the treatment region, cells located in the focal volume may be rapidly heated while intervening and surrounding tissues outside the focus are spared the same level of heating. Surrounding tissues are unaffected or affected less in the unfocused portion of the ultrasound beam because the energy is spread over a correspondingly larger area. The transmitted HIFU pulses have the determined frequency, power dose, or other characteristic.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for high intensity ultrasound path determination, the method comprising:
identifying a plurality of possible paths for high intensity ultrasound from one or more transducers to a treatment region within a patient;
selecting a subset of the plurality of possible paths as a function of ultrasound response; and
transmitting the high intensity ultrasound along the paths of the selected subset.

2. The method of claim 1 wherein identifying the plurality of possible paths comprises transmitting along a plurality of scan lines corresponding to each of the possible paths.

3. The method of claim 1 wherein identifying the plurality of possible paths comprises identifying the possible paths through a volume, data representing the volume being acquired without regard to the possible paths.

4. The method of claim 1 wherein identifying the plurality of possible paths comprises identifying at least one possible path for each of at least two separate therapy transducers.

5. The method of claim 4 wherein selecting comprises selecting the possible paths free of obstruction from metal and bone.

6. The method of claim 1 wherein selecting the subset comprises selecting as a function of distance along each possible path from the one or more transducers to the treatment region.

7. The method of claim 1 wherein selecting the subset comprises selecting as a function of an obstruction, the possible paths intersecting an obstruction not being selected.

8. The method of claim 1 wherein selecting the subset comprises selecting as a function of a sensitive region, the possible paths intersecting a sensitive region not being selected.

9. The method of claim 1 further comprising:
adapting a frequency of the high intensity ultrasound as a function of depth from the transducer to the treatment region, attenuation characteristic along the path, or combinations thereof.

10. The method of claim 1 further comprising:
determining a power dose of the high intensity ultrasound along each of the paths in the subset, the power dose being a function of tissues along the path, distance from the transducer to the treatment region along the path, number of paths in the subset, frequency of the transmission, or combinations thereof.

11. A system for high intensity ultrasound path determination, the system comprising:
at least one therapy transducer operable to transmit high intensity focused ultrasound;
at least one imaging transducer operable to transmit acoustic energy for imaging; and
a processor operable to determine an origin of a beam of the high intensity focused ultrasound relative to a treatment region, the origin determined as a function of patient characteristics between origin options and the treatment region, and the processor being operable to determine the patient characteristics and origin as a function of data received with the imaging transducers.

12. The system of claim 11 wherein the at least one therapy transducer comprises a multidimensional array of elements;
further comprising:
a transmit beamformer operable to generate relatively delayed electrical signals establishing the origin at a location on the multidimensional array.

13. The system of claim 11 wherein the at least one therapy transducer comprises a plurality of therapy transducers, and wherein the processor is operable to determine the origin as from one of the plurality of therapy transducers and not from another of the plurality of therapy transducers.

14. The system of claim 11 wherein the at least one therapy transducer and the at least one imaging transducer comprises a blanket having a plurality of therapy transducers and a separate plurality of imaging transducers, and wherein the processor is operable to select the therapy transducers without an obstruction along a scan line from the therapy transducer to the treatment region, the origin corresponding to one of the scan lines.

15. The system of claim 11 wherein the at least one therapy transducer and the at least one imaging transducer are a same transducer.

16. The system of claim 11 wherein the processor is operable to identify bone or metal as the patient characteristic and determine the origin to avoid transmitting the high intensity focused ultrasound through the bone or metal.

16. The system of claim 11 wherein the at least one therapy transducer comprises a transducer operable from outside of a patient.

17. The system of claim 11 wherein the processor is operable to determine a power and frequency as a function of the patient characteristic between the origin and the treatment region.

18. In a computer readable storage medium having stored therein data representing instructions executable by a programmed processor for high intensity ultrasound path determination, the storage medium comprising instructions for:
transmitting acoustic energy along a plurality of scan lines;
receiving signals responsive to the transmitting; and
optimizing, for high intensity focused ultrasound, a path to a region to be coagulated, the optimizing being a function of the received signals.

19. The instructions of claim 18 wherein optimizing the path comprises determining a plurality of possible scan lines, and selecting at least one scan line of the plurality of possible scan lines associated with avoiding an acoustic obstruction.

20. The instructions of claim 18 wherein transmitting and receiving comprise forming a dataset representing a three-dimensional volume;
further comprising:
identifying the region to be coagulated;
wherein optimizing comprises examining data along rays cast from available sources of the high intensity focused ultrasound through the three-dimensional volume to the region to be coagulated, and selecting rays avoiding an acoustic obstruction, a heat sensitive region, or combinations thereof, the selecting being, at least, a function of the data along the rays; and
further comprising:
transmitting the high intensity focused ultrasound along the selected rays, the acoustic energy having less power than the high intensity focused ultrasound.

21. The instructions of claim 20 wherein forming comprises aligning and combining volumes associated with different imaging transducers, the aligned and combined volumes comprises the three-dimensional volume, wherein examining data along rays comprises examining data from the dataset from therapy transducers to the region to be coagulated, the spatial position of the therapy transducers relative to the imaging transducers being known or measured.

22. The instructions of claim 18 wherein transmitting and receiving comprises transmitting and receiving along scan lines intersecting the region to be coagulated and from available sources of the high intensity focused ultrasound, and wherein optimizing comprises examining data representing the scan lines, and selecting scan lines avoiding an acoustic obstruction, a heat sensitive region, or combinations thereof, the selecting being a function of the data along the scan lines;
further comprising:
transmitting the high intensity focused ultrasound along the selected scan lines, the acoustic energy having less power than the high intensity focused ultrasound.

23. The instructions of claim 18 further comprising:
determining a power and frequency of the high intensity focused ultrasound as a function of a characteristic of the path.
